(19) United States
(12) Patent Application Publication
(10) Pub. No.: US 2002/0009800 A1
(43) Pub. Date: Jan. 24, 2002

(54) Virus vectors and expression elements

(76) Inventors: Stacey Efstathiou, Cambridge (GB); Robin Henry Lachmann, Cambridge (GB)

Correspondence Address: KLARQUIST SPARKMAN CAMPBELL, LEIGH & WHINSTON, LLP, One World Trade Center, Suite 1600, 121 S.W. Salmon Street, Portland, OR, 97204, US

(21) Appl. No.: 09/804,691

(22) Filed: Mar. 12, 2001

Related U.S. Application Data
(63) Continuation of application No. 09/365,662, filed on Aug. 2, 1999, now abandoned.

(30) Foreign Application Priority Data
Jul. 31, 1998 (GB) 9816770.3
Jul. 31, 1998 (GB) 9816773.7

(51) Int. Cl. C12N 7/01; C12N 15/86

(52) U.S. Cl. 435/320.1; 435/235.1; 435/456

(57) ABSTRACT

A mutant herpesvirus has inactivating (preferably deletion) mutations at the locus of both native copies of the latency-active regulatory sequences. The resulting virus can be used as a latency-inactive virus as the basis of vectors for gene delivery, as a helpervirus for production of amplicons, and as a base virus mutant for the construction of mutant virus vectors carrying synthetic latency-active regulatory sequences. Also described are synthetic/semisynthetic latency-active regulatory sequences and their use in CNS and other cells.

### FIELD OF THE INVENTION

This invention relates to herpesviral vectors with altered latency-active expression elements, suitable for use in connection with for example therapeutic gene delivery. The invention also relates to nucleic acid constructs comprising such expression elements, to methods for the construction and use of such nucleic acids and vectors, and to compositions comprising them. Among other things the invention provides synthetic/semisynthetic latency-active regulatory sequences and virus vectors based on them and their use in CNS and other cells.

### BACKGROUND OF THE INVENTION

Mutant herpesviruses with modified latency-active expression elements are known.

Thus, for example, international patent application WO 97/20935 (CU Tech Services Ltd: Efstathiou & Lachmann), describes latency-active herpesviral expression elements comprising a latency-active promoter and an IRES as well as a heterologous gene to be expressed by the help of the promoter and IRES.

Also, international patent application WO 96/27672 (Fink & Glorioso) describes a mutant herpes simplex virus type 1, which is attenuated or replication defective, and comprises: (a) a latency active promoter having a LAP2 sequence and (b) a non-herpes simplex virus type 1 gene encoding a protein operatively connected to the latency active promoter, such that the gene is expressed to produce the non-herpes protein in biologically active form by a cell latently infected by the virus, and discloses use of the modified herpes simplex virus for purposes of gene therapy to express a gene of interest, esp. a neurotrophic factor (pref. nerve growth factor, ciliary neurotrophic factor, brain derived neurotrophic factor, glial derived neurotrophic factor or neurotrophin-3), a neurotrophic factor receptor, preproenkephalin, a superoxide dismutase or an androgen receptor in dorsal root ganglion cells of the sensory nervous system and in anterior horn cells of the motor nervous system, when these cells are latently infected by the viral vector.

Further publications relating to (or identifying) herpesviral latency-active regulatory DNA regions include R H Lachmann et al, J Virol 1997 71(4) 3197-3207; J L Arthur et al, J Gen Virol (1998) 79: 107-116; and W F Goins et al, J Virol (1994) 68(4):2239-2252.

Further references relevant e.g. to herpesviral latency include:

Batchelor, A. H. and P. O'Hare (1990). Regulation and cell-type-specific activity of a promoter located upstream of the latency-associated transcript of herpes simplex virus type 1. J Virol 64, 3269-3279.

Carpenter, D. E. and J. G. Stevens (1996). Long-term expression of a foreign gene from a unique position in the latent herpes simplex virus genome. Human Gene Therapy 7, 1447-1454.

Ecob-Prince, M. S., K. Hassan, M. T. Denheen and C. M. Preston (1995). Expression of beta-galactosidase in neurons of dorsal root ganglia which are latently infected with herpes simplex virus type 1. J Gen Virol 76, 1527-1532.

Forrester, A., H. Farrell, G. Wilkinson, J. Kaye, P. N. Davis and T. Minson (1992). Construction and properties of a mutant of herpes simplex virus type 1 with glycoprotein H coding sequences deleted. J Virol 66, 341-348.

Harris, R. A. and C. M. Preston (1991). Establishment of latency in vitro by the herpes simplex virus type 1 mutant in1814. J Gen Virol 72, 907-913.

Herz, J. and R. D. Gerard (1993). Adenovirus-mediated transfer of low density lipoprotein receptor gene acutely accelerates cholesterol clearance in normal mice. Proceedings of the National Academy of Sciences 90, 2812-2816.

Jamieson, D. R., L. H. Robinson, J. I. Daksis, M. J. Nicholl and C. M. Preston (1995). Quiescent viral genomes in human fibroblasts after infection with herpes simplex virus type 1 Vmw65 mutants. J Gen Virol 76, 1417-1431.

Kaplitt, M. G., A. D. Kwong, S. P. Kleopoulos, C. V. Mobbs, S. D. Rabkin and D. W. Pfaff (1994). Preproenkephalin promoter yields region-specific and long-term expression in adult brain after direct in vivo gene transfer via a defective herpes simplex viral vector. Proc Natl Acad Sci U S A 91, 8979-8983.

Kim, D. W., T. Harada, I. Saito and T. Miyamura (1993). An efficient expression vector for stable expression in human liver cells. Gene 134, 307-308.

Kim, D. W., T. Uetsuki, Y. Kaziro, N. Yamaguchi and S. Sugano (1990). Use of the human elongation factor 1 alpha promoter as a versatile and efficient expression system. Gene 91, 217-223.

Lokensgard, J. R., D. C. Bloom, A. T. Dobson and L. T. Feldman (1994). Long-term promoter activity during herpes simplex virus latency. J Virol 68, 7148-7158.

Miyanohara, A., P. A. Johnson, R. L. Elam, Y. Dai, J. L. Witztum, I. M. Verma and T. Friedmann (1992). Direct gene transfer to the liver with herpes simplex virus type 1 vectors: transient production of physiologically relevant levels of circulating factor IX. New Biol 4, 238-246.

Wu, N., S. C. Watkins, P. A. Schaffer and N. A. DeLuca (1996). Prolonged gene expression and cell survival after infection by a herpes simplex virus mutant defective in the immediate-early genes encoding ICP4, ICP27, and ICP22. Journal of Virology 70, 6358-6369.

In addition, A T Dobson et al (Neuron, 1990, 5:353-360) disclosed a HSV mutant which besides being negative for ICP4 function, had small deletions within the LAT-encoding region to disable LAT function, and the mutant virus also was reported to carry a transgene under control of a promoter from MMLV virus. This virus was reported to bring about short-term gene expression when used to infect cells within the CNS.

It remains desirable to provide further latency-active expression elements for herpesviruses, and corresponding mutant viruses and methods and compositions for preparing and using them. The present inventors also believe that the use of the natural herpesviral latency-active promoters to secure longterm gene expression from the latent viral state can be associated with a drawback due to relative weakness of the natural viral promoter, and a further aim is to provide expression constructs and vectors to ameliorate this drawback.

For several purposes in this specification it is convenient to refer to nucleotide positions of the genome sequence of HSV1 strain 17 as published by D McGeoch 1988 (J Gen Virol 69:1531-1574), D McGeoch 1991 (J Gen Virol 72:3057-3075, and Perry and McGeoch 1988 (J Gen Virol 69:2831-2846. Such references are not intended to be restricted to the particular genome sequence so published, but include references to homologous positions of related herpesviruses, and the exact limits of regions so referred to are generally not critical for the purposes of this invention.

### SUMMARY AND DESCRIPTION OF THE INVENTION

The present invention provides in one aspect a mutant herpesvirus in which both native copies of the latency-active regulatory sequences (which normally enable longterm expression in the latency-active state) have been inactivated by deletion mutations, e.g. deletion of substantially the entire native latency-active regulatory sequences. Such deletion can be of the latency-active regulatory region as defined in the publications mentioned above or any of them, and can be carried out using per-se known DNA manipulation techniques. For example, the entire LAT (i.e. LAT regulatory) region of about 3 to 3.4 kb can be deleted in both native copies, so as to produce a viral mutant that is stable in the sense that it can be propagated without appreciable recombination.

The resulting mutant herpesvirus can be used for several purposes: e.g. as a helper virus for the production of amplicons (e.g. amplicons made according to WO 96/29421, publ Sep. 26, 1996, Lynxvale Ltd & Cantab Pharmaceuticals Research Ltd: S Efstathiou, S C Inglis, X Zhang); as a latency-inactive virus for the construction of vectors carrying heterologous DNA, e.g. encoding gene products of any of the kinds mentioned in WO 96/26267 (Cantab Pharmaceuticals Research: MEG Boursnell et al), or in WO 96/27672 cited above, that are to be delivered to target cells; and as a base virus mutant for the construction of mutant virus vectors carrying synthetic (e.g. sernisynthetic) latency-active regulatory sequences. Heterologous genes that can be included as ‘cargo’ genes in virus vectors according to examples of the invention can encode for example products selected from neurotrophic factors, such as GDNF, CTNF, and BDNF, and nerve growth factors such as NGF. Further examples of useful ‘cargo’ genes are those encoding hexosaminidase (known in connection with Tay-Sachs and Sandhoff diseases), arylsulphatase A (known in connection with metachromatic leucodystrophy), the NPC1 gene (known in connection with Niemann Pick Disease type C) and glucocerebrosidase (known in connection with Gaucher's disease).

In another aspect, the invention provides a mutant herpesvirus in which (a) both native copies of the latency-active (LA) regulatory sequences (which normally enable longterm expression in the latency-active state) have been inactivated by deletion mutations, e.g. deletion of substantially the entire native latency-active regulatory sequences; and in which (b) a latency-active regulatory sequence has been inserted e.g. at a locus other than one of the natural LA loci.

The latency-active sequence can for example comprise a synthetic or semisynthetic latency-active regulatory sequence, e.g. based on a sequence made according to international patent application WO 97/20935 (CU Tech Services Ltd: Efstathiou & Lachmann), comprising a latency-active promoter and an IRES as well as a heterologous gene to be expressed by the help of the promoter and IRES. Alternatively, the latency-active sequence can be a latency-active sequence as described in WO 96/27672 (Fink D J, Glorioso J C).

Such a mutant herpesvirus can also be if desired for example a genetically disabled mutant virus in which a gene essential for production of infectious new virus particles has been inactivated, preferably by deletion. The inserted latency active regulatory sequence can preferably be inserted at the site of deletion of the essential gene, e.g. a gene encoding an essential glycoprotein for example gH.

A synthetic or semisynthetic latency-active promoter can for example be made for present purposes according to examples of the present invention using either of two well characterised promoter elements, for example. One is the human cytomegalovirus immediate early (HCMV IE) promoter, a viral promoter known to be strongly active in the context of the HSV genome during acute infection (Forrester et al., 1992), and shown to drive some transcription from the latent genome (Ecob-Prince et al., 1995). Another is the human elongation factor 1a (EF-1a) promoter, strongly transcriptionally active in a large range of cell types tested in vitro (including neuronal and lymphoid cells), that gives high expression of CAT in all tissues of a transgenic mouse (Kim et al., 1990), and that has been shown to give efficient and stable expression in a human hepatocyte cell line (Kim et al., 1993). Such a promoter can be inserted either into the LAT region, where they can be linked to potential LAP-derived long-term elements, and/or into a non-essential locus such as the US5 locus, which is situated conveniently well away from the repeat regions of the viral genome.

Mutant viruses according to this aspect of the invention can be used as gene delivery vectors with a useful safety feature, see also WO 96/26267, cited above.

The invention provides in a further aspect a setnisynthetic herpesviral latency-active gene promoter DNA sequence comprising in the 5′-3′ direction (a) a core promoter element other than a natural herpesviral latency-active core promoter element, (b) a longterm expression element, e.g. an approx 1.5 kb herpesviral latency-active sequence from downstream of the natural core LAP, and (c) an IRES: when the promoter is applied in a gene delivery vector this sequence is normally followed by a gene to be expressed in a target cell. In certain examples the latency-active property can be conferred or enhanced by a LAP regulatory element located upstream of the heterologous core promoter element (a) and comprising sequence(s) contained within a 1.6 kb region corresponding to nucleotides 117010-118664 in the HSV genome as published by McGeoch 1988, McGeoch 1991 and Perry and McGeoch 1988.

Also provided are herpesviral vectors containing such a semisynthetic promoter sequence in assocation with a heterologous DNA sequence to be expressed in a target cell. Among useful examples of the invention are non-lytic mutant herpesviral vectors carrying the promoter sequences.

In this connection, according to a further aspect of the present invention, the present inventors have dissociated the long-term activity of the LAP from its neuronal-specific elements to obtain reporter gene expression in latently infected tissue culture cells. Thus the invention provides among other things synthetic/semisynthetic latency-active regulatory sequences and virus vectors based on them and their use in CNS and other cells, and the use of mutant viruses as described herein for gene delivery, not only for expression of genes in cells of the central nervous system that are latently infected by said mutant virus, but also in non-CNS latently infected cells.

The core promoter element can advantageously be a strong promoter of known type, for example a CMV-IE core promoter element, or it can be a cell-specific core promoter element such as for example a mammalian-tissue-specific core promoter element.

For example the core promoter element used for present purposes can be either of two well characterised promoter elements. The first is the human cytomegalovirus immediate early (HCMV IE) promoter, a viral promoter known to be strongly active in the context of the HSV genome during acute infection (Forrester et al., 1992), and shown to drive some transcription from the latent genome (Ecob-Prince et al., 1995). The second is the human elongation factor 1a (EF-1a) promoter, strongly transcriptionally active in a large range of cell types tested in vitro (including neuronal and lymphoid cells), that gives high expression of CAT in all tissues of a transgenic mouse (Kim et al., 1990), and that has been shown to give efficient and stable expression in a human hepatocyte cell line (Kim et al., 1993). Such a promoter can be inserted either into the LAT region, where they can be linked to potential LAP-derived long-term elements, and/or into a non-essential locus such as the US5 locus, which is situated conveniently well away from the repeat regions of the viral genome, or (preferably in some cases) into an essential locus e.g. the site of a (deleted) gH gene of HSV.

The semisynthetic promoter sequence can for example be made in-situ in a mutant herpesvirus by excising an approx 300bp core promoter element from the LAP (e.g. in one example a 203-bp PstI fragment of HSV1 encompassing the LAT transcription start site and elements of the basal promoter), and replacing this with a CMV-IE or other strong core promoter element, e.g. an element that is not substantially homologous with the element excised.

Such promoter elements and virus vectors containing them can be used to achieve superior levels and durations of heterologous gene expression in host cells of a variety of types: in neuronal cells and in non-neuronal cells. With known non-lytic mutant herpesviral vectors, non-lytic infection of non-neuronal host cells has been associated with less than desired levels and durations of expression of heterologous genes and thew present invention can be used to give good levels and durations of expression.

The following description is given for illustration and not for limitation to show particular enbodiments of the invention.

LAT-deletant HSV-1

An example of an HSV-1 mutant virus containing a deletion of the LAT region, in both the IRL and TRL copies of the LAT sequences, can for example be constructed by the use of a plasmid containing a 10.1 kb BamH I B fragment from HSV-1 strain SC-16. (This fragment corresponds to nucleotides 113322-123459 of the published genome sequence of HSV1 strain 17, see references cited above). This sequence includes a 3.3 kb Hpa I fragment (nucleotides 117010-120301) encompassing the IRL copy of the LAP. The 10.1 kb fragment can for example be cloned in a proprietary plasmid system such as pBluescribe M13-. A plasmid containing a modified version of this 10.1 kb fragment, in which the 3.3 kb fragment encompassing the LAP has been deleted, can be made either by complete HpaI digestion (followed by isolation and re-ligation of a 1.2 kb additional HpaI fragment adjacent to but outside the LAP-containing 3.3 kb HpaI fragment), or by partial Hpa I digestion, and purification of the wanted deletion product. The resulting deletion plasmid can be used for recombination with virus by per-se standard technique. The recombination process can yield LAT-deleted virus in either one or two stages. In a single stage, the recombination products can be screened for low-frequency virus recombinants in which both copies of the LAT region have been deleted. An example of a LAT-deletant virus constructed in this way is given by S.Cai (in Ph.D. dissertation, Hughes Hall, University of Cambridge, April 1999). Alternatively, in two stages, the recombination can first provide a first-stage LAT deletant herpesvirus which lacks the IRL copy of the LAT region, but retains the TRL copy of the LAT region. This deeltant can then be used for further recombination, to form a second stage LAT deletant, lacking both copies of the LAT region, by use of a further deletion plasmid constructed in analogous manner to the plasmid indicated above, but incorporating, instead of the IRL-homologous sequence as indicated above, a viral genome sequence fragment homologous with the sequence around the TRL copy of the LAT region, and deleted in respect of the sequence of the TRL copy of the LAT region.

HSV-1 Mutants Containing Semisynthetic Latency-Active Promoter

(a) An example of a virus vector according to another embodiment of the invention, designated L beta E, can be constructed so as to be similar to L beta A (designating a virus vector described in WO 97/20935, CU Tech Services Ltd: S Efstathiou & R H Lachmann, incorporated herein by reference), and can contain all potential downstream and upstream long-term LAP regulatory regions linked to the IRES-lacZ cassette; except that L beta E can have modifications in that that a Pst I fragment (nucleotides 118664 to 118867), which contains the transcriptional start site for the primary LAT transcript, is replaced by a fragment containing the HCMV IE promoter (extending from nucleotide −299 to +69 with respect to the CMV IE1 transcript start site), such that the HCMV IE promoter drives transcription in the direction of the IRES-lacZ cassette.

Such a virus is expected to show good gene expression for extended periods within murine sensory neurones.

(b) A further example of a virus vector according to an aspect of the invention can be constructed so as to be similar to L beta A (designating a virus vector described in WO 97/20935, CU Tech Services Ltd: S Efstathiou & R H Lachmann, incorporated herein by reference), and can contain all potential downstream and upstream long-term LAP regulatory regions linked to the IRES-lacZ cassette, except that it can have modifications as follows:- (a) the deletion of both copies of the latency active regulatory region as described above, and (b) insertion of a sequence elsewhere in the genome, as described above, the insert corresponding to the latency active region of the virus of WO 97/20935 except that a Pst I fragment (nucleotides 118664 to 118867), which contains the transcriptional start site for the primary LAT transcript, has been replaced by a fragment containing the HCMV IE promoter (extending from nucleotide −299 to +69 with respect to the CMV IE1 transcript start site), such that the HCMV IE promoter drives transcription in the direction of the IRES-lacZ cassette.

(c) Alternative vectors according to the invention can for example comprise similar expression elements (ie HCMV E promoter and IRES-lacZ cassette) and deletions as described herein, in a vector that has a fully replication-defective ‘background’ (such as an in1814 mutant (see Harris and Preston, 1991) or a virus vector according to U.S. Pat. No. 5,658,724, Univ Pittsburgh: N A DeLuca) carrying plural deletions of immediate early genes encoding ICP4 and ICP27, and can be applied to uses in which either neuronal or non-neuronal cells are infected. The following example and experiments conducted therewith indicate that virus vectors according to the invention can be made so as to give latent expression over extended periods of time in a variety of cell types not limited to CNS cells.

Construction and Use of Vector in1378:

Use is made of insertion of an internal ribosomal entry site (IRES)-linked transgene into a HpaI locus (nt. 120301-120469) situated 1.5 kb downstream of the mLAT transcription start site, in the recombinant virus SC16 LβA, to procure transgene expression within latently infected neurones (WO 97/20935, Lachmann & Efstathiou, 1997, Lachmann et al., 1999). This transgene expression has been found to have similar kinetics to wild-type LAT expression and retention of the 1.5 kb of sequence which lies downstream of the latency-associated promoter (LAP) transcription start site is used as one way to preserve all the elements required for authentic LAP activity.

For in1378, an LAP based expression construct was incorporated on to a highly replication defective ‘backbone’, virus in1312. Virus in1312 has mutations in the VP16, ICP0 and ICP4 genes (Preston et al., 1998). Viruses constructed on this backbone can show minimal cytotoxicity and, after high multiplicity infection, can establish a latent infection in monolayers of tissue culture cells.

Mutant in1388 was constructed as a derivative of in1312 which expresses βGeo under LAP control. Virus in1388 can establish latency efficiently after footpad injection and can give abundant βgal expression in latently infected neurones. However, in1388 appears not to express βGEO in latently infected monolayers of tissue culture cells and it appears that latent-phase LAP activity here is neuronal-specific.

According to a further aspect of the present invention, the present inventors have dissociated the long-term activity of the LAP from its neuronal-specific elements to obtain reporter gene expression in latently infected tissue culture cells.

As a vector for this purpose, mutant in1378 was constructed. This virus is homologous to in1388, but the core LAP and transcription start site were replaced with a minimal HCMV IE promoter and transcription start site. This resulted in a virus in which βGEO was under the transcriptional control of a hybrid HCMV IE/LAP promoter. This hybrid promoter contains a strong basal promoter with no cell-type specificity, as well as the 1.5 kb of LAP sequence downstream of the transcription start site which is believed to confer or enhance long-term LAP activity. The kinetics of βGEO expression from these viruses was tested in latently infected neurones in vivo and in vitro and in monolayers of Vero cells.

In further detail, the viral structures under consideration here are as follows: References relating to these viral constructs are as follows:

Ace, C. I., et al. (1989). Construction and characterization of a herpes simplex virus type 1 mutant unable to transinduce immediate-early gene expression. Journal of Virology 63, 2260-9.

Lachmann, R. H., et al. (1997). Utilization of the Herpes Simplex Virus type-1 latency-associated regulatory region to drive stable reporter gene expression in the nervous system. Journal of Virology 71, 3197-3207.

Lachmann, R. H., et al. (1999). An analysis of Herpes Simplex virus gene expression during latency establishment and reactivation. Journal of General Virology 80, 1271-1282.

Preston, C. M., et al. (1998). Herpes simplex virus type 1 immediate early gene expression is stimulated by inhibition of protein synthesis. Journal of General Virology 79, 117-24.

in1312: Virus in1312 (Preston et al., 1998) has a mutation in the gene for the virion transactivator protein (VP16) which abolishes its ability to transactivate expression of the five viral immediate early (IE) genes (Ace et al., 1989). In addition ICP0 is non-functional (the RING finger has been deleted) and there is a temperature-sensitive mutation in the ICP4 gene (tsK). This virus can only be propagated in BHK cells (which complement for the ICP0 defect) in the presence of HMBA (a chemical agent which compensates for the VP16 defect) at the permissive temperature of 31° C. In non-permissive conditions (at 37 deg.C the tsk mutation is thought to be ‘leaky’ and attenuating) this virus is non-cytotoxic and it can persist in tissue culture cells after infection at high multiplicity.

in1382: This is a mutant virus constructed by inserting a cassette consisting of the HCMV IE promoter (nucleotides −750 to +5 with regard to the transcription start site) linked to lacZ into the TK locus of in1312.

in1388: Virus in1388 has been constructed to allow latent-phase expression of βgal in latently infected neurones. A cassette consisting of the EMCV IRES linked to a lacZ-neo^{R} fusion gene (βGEO) has been inserted into the in 1312 genome at the Hpa I locus which lies 1.5 kb downstream of the mLAT transcription start site. This virus is therefore analogous to the virus SC16 LβB (Lachmann & Efstathiou, 1997) and is capable of directing long-term transgene expression in latently infected sensory neurones.

in1378: This virus, according to a useful form of the persent invention, has been derived from in1388. A PstI fragment incorporating the core LAP and mLAT transcription start site (nucleotides 118659-118862) has been deleted from the in1388 genome and replaced by the HCMV IE promoter (nucleotides −299 to +67 with regard to the HCMV IE transcription start site). In this virus, expression of βGEO (as an example of a ‘cargo’ gene for delivery by use of the virus as a vector) is therefore under the control of a semisynthetic hybrid HCMV IE/LAP promoter.

Latent-phase transgene expression was studied after inoculation of mouse footpad with in1378:

Mice were infected by injection of 3×10⁵ pfu of either in1388 or in1378 into the left footpad. At each time point, three mice were sacrificed and the lumbar dorsal root ganglia were histochemically stained for βgal activity. The average number of βgal positive neurones per mouse for each time point is shown in the table below.

| | in1388 | in1378 |
|---|---|---|
| Day 4 | 24 | 29 |
| Day 12 | 52 | 38 |
| Day 26 | 62 | 43 |
| Day 56 | 61 | 32 |

These data show that the hybrid HCMV IE/LAP promoter present in in1378 was capable of mediating latent-phase transgene expression in neurones with kinetics similar to the authentic LAP present in in1388. This indicates that there are sequence elements present in the regions upstream and/or downstream of the core LAP which can confer transcriptional activity onto a heterologous basal promoter in latently infected sensory neurones in vivo.

At the 56 day time point the footpads of latently infected mice were dissected and stained for βgal activity. No βgal activity was detected in the footpads of mice latently infected with in1388 but blue staining was seen in the foot muscles of the mice infected with in1378. Microscopy indicated that this staining was related to the end plates of motor axons innervating the site of inoculation. This implies that there were latently infected motor neurones within the spinal cords of the in1378-treated mice which were expressing large amounts of βgal and exporting it to the periphery. This peripheral expression of βgal was not seen in the in1388 infected mice. This implies that the hybrid HCMV IE/LAP promoter construct was functioning more efficiently in spinal cord motor neurones here than the endogenous LAP.

Latent-phase transgene expression was examined in cultured sensory neurones:

Primary cultures of sensory neurones were established from dorsal root ganglia harvested from newborn rat pups. Established cultures, containing approximately 600 neurones, were infected with 10⁶ pfu of in1382, in1378 or in1388. Cultures were fixed at 3, 7 and 20 days post-infection. Cultures were stained immunohistochemically for βgal and β-tubulin (a neurone-specific marker). At each time point representative fields from the whole cover slip were examined and the total number of neurones and number of βgal expressing neurones counted. The table below shows the percentage of βgal-positive neurones detected for each virus at each time point.

| | Day 3 | Day 7 | Day 29 |
|---|---|---|---|
| in1382 | 73% | 91% | 13% |
| in1388 | 56% | 67% | 54% |
| in1378 | 72% | 85% | 38% |

The number of in1382 infected neurones expressing βgal was seen to decrease from day 7 to day 20 as it is believed the HCMV IE promoter became transcriptionally silenced. With both viruses in1388 and in1378 there were still significant numbers of βgal expressing neurones at the day 20 time point (it should be noted that there were considerably fewer neurones left on each cover slip at this time point than at the acute time points).

In cultures infected with in1378 it was possible to detect βgal expressing non-neuronal cells at the day 7 and day 20 time points. Similar cells were not seen at late time points after infection with in1382 and were never seen after infection with in1388. These data indicate that the hybrid HCMV IE/LAP promoter was capable of remaining transcriptionally active in latently infected non-neuronal cells.

Latent-phase transgene expression was examined in cultured Vero cells:

Monolayers of Vero cells on 6 well dishes were infected with 5×10⁶ pfu of either in1382, in1378 or in1388. On days 1, 3 and 6 post infection monolayers were fixed and histochemically stained using X-gal. Stained monolayers were examined. All monolayers appeared to be healthy, indicating that these in312-based vectors were not cytotoxic. After infection with in1382, there was abundant βgal expression from the HCMV IE promoter on day 1, but this rapidly diminishes as the promoter is believed to ahve become transcriptionally silenced. With in1388, the endogenous LAP was not transcriptionally active in these cells at any time after infection. In monolayers infected with in1378, βgal expression from the hybrid HCMV IE/LAP promoter increased over time. Therefore the LAT region sequences which flank the HCMV IE promoter in this virus were able to prevent it becoming transcriptionally silenced during the first six days of latency in these non-neuronal cells.

These experiments are believed to show among other things that embodiments of the invention utilising elements of the HSV LAP together with a heterologous basal promoter possessed latent-phase activity. It is believed that a hybrid HCMV IE/LAP construct, provided by examples of the invention, can be active in a wider range of latently infected neuronal types than the endogenous LAP. In particular, such a hybrid HCMV IE/LAP construct can mediate reporter gene expression in some latently infected non-neuronal cell types.

These examples illustrate a general strategy according to the invention. Significant elements are for example use of an IRES lacZ cassette inserted about 1.5 kb downstream of the LAT transcription start site, as in patent application WO 97/20935 cited above, in order to maintain all long-term expression elements, and the addition of heterologous promoter elements at or around the LAT transcription start site in order to increase basal transcription levels, and to extend tissue specificity. Although a virus mutant made in this way has the core LAP and LAT transcription start site deleted, this is not necessary for the present invention. It would for example also be possible to simply insert other promoter elements into this region, and have two different transcription start sites, or indeed to insert enhancer elements, or similar sequences, which would act to increase the activity or tissue specificity of the core LAP itself.

As well as using strong, non-tissue specific promoters like the HCMV IE promoter or the human EF1-a promoter discussed above, one can also envisage the use of tissue specific promoters to restrict expression to certain tissues such as liver (the human albumin promoter) or muscle (some suitable muscle specific promoter), or to certain groups of cells (ie use the tyrosine hydroxylase promoter to restrict expression to dopaminergic neurones). An exhaustive list of the possible uses would be very long indeed, as can be appreciated by a reader skilled in the art.

In a further aspect of the invention, a cassette containing such a combinatorial promoter can be moved or placed into other loci within the viral genome (especially for example on a LAT-deleted ‘backbone’), or into an amplicon, or into other forms of expression system. This corresponds for example to analogous uses set out in relation to the basic LAP promoter in WO 97/20935 cited above. Similarily, the list of available uses here is vast, and the promoter allows expression of any of a variety of desired “payload” genes in any tissue. Many forms of somatic cell gene therapy are thereby made available, e.g. those mentioned in WO 97/20935 and other documents cited therein and hereinabove.

A virus according to an example of the invention can have deletions in both copies of the LAT promoter region in the repeats flanking the UL region of the genome and comprises a 3.3 kb HpaI fragment containing an LAT promoter-containing sequence modified by attachment (at the 3′end) of an IRES sequence and a cargo gene cassette, e.g. for experimental purposes a lacz cassette. For other purposes the cargo gene can be any gene that it is desired to deliver to cells which are to be infected by the modified virus, e.g. cells in culture, cells ex-vivo, or cells in-vivo.

This promoter-reporter gene (or promoter-cargo gene) cassette can be inserted in any desired position of the mutant herpesviral genome, e.g. a non-essential site such as the US5 locus of HSV-1 strain SC-16, but more preferably an essential site, such as the site of deletion of an essential viral gene such as the gH gene of HSV. In the latter case the mutant viruses are cultured for production purposes on cell lines made recombinant to complement the function of the missing essential viral gene, e.g. gH of HSV.

By the use of viruses constructed in this way it has been confirmed that the the 3.3 kb HpaI fragment, after deletion from its native locus, and insertion as part of a promoter-IRES-cargo gene cassette elsewhere in the HSV genome, is effective in conferring a promoter function derived from the LAT promoter which mediates longterm expression within neurones. Such a promoter cassette can also be inserted to operate in other viral vector systems, including HSV-derived amplicons, adenovirus vectors, and retrovirus-based systems.

Thus it can be seen that the invention provides inter alia a variety of mutant herpesviruses and virus vectors (a) with synthetic latency-active expression elements and/or (b) with no latency-active expression elements: and that the invention also provides a variety of latency-active viral gene promoters and vectors containing them, e.g. for the delivery genes and expression of gene products as mentioned herein and the references given herein.

The invention is susceptible to a variety of modifications and variations as will be apparent to those skilled in the art, and the present disclosure extends to combinations and subcombinations of the features mentioned or described herein and in the cited documents which are hereby incorporated by reference in their entirety for all purposes.

1. A mutant herpesvirus in which both copies of the latency-active regulatory sequences (which normally enable longterm expression in the latency-active state) have been completely or substantially completely deleted.

2. A mutant herpesvirus according to claim 1 in which a non-native latency-active regulatory sequence has been inserted elsewhere than at a normal locus of the latency active regulatory sequence.

3. A mutant herpesvirus according to claim 2, in which the non-native latency-active regulatory sequence comprises a modifed sequence derived from the corresponding native latency-active regulatory sequence.

4. A mutant herpesvirus according to claim 3, wherein the non-native latency-active regulatory sequence comprises a modified sequence derived from the corresponding native latency-active sequence by insertion of a heterologous promoter and a heterologous gene encoding a desired expression product.

5. A mutant virus according to claim 2, wherein the inserted semi-synthetic latency active regulatory sequence comprises (a) a promoter element other than the native herpesviral latency active core promoter, (b) a longterm expression element, (c) an internal ribosome entry site, and (d) a heterologous gene sequence arranged so that the gene expression is under control of the synthetic or semi-synthetic latency-active regulatory sequence.

6. A mutant virus according to claim 4, wherein the heterologous promoter is a CMV-IE promoter.

7. A mutant virus according to claim 4, wherein the heterologous gene encodes a product selected from neurotrophic factors and nerve growth factors.

8. A mutant virus according to claim 2, wherein the non-native sequence has been inserted at the locus of a deleted viral gene essential for production of infectious new viral particles.

9. A mutant virus according to claim 8, wherein the locus is that of an essential viral glycoprotein such as gH.

10. Use of a mutant virus according to claim 1 or 2 for gene delivery, e.g. for expression of said gene from latency of infection by said mutant virus in a cell of the central nervous system (CNS) or in a non-CNS cell.
